(19) United States
(12) Patent Application Publication
(10) Pub. No.: US 2021/0122870 A1
(43) Pub. Date: Apr. 29, 2021

(54) AQUEOUS DISPERSION OF A POLYURETHANE COMPRISING A DICARBOXYLATE WITH ONE OR TWO SECONDARY AMINO GROUP

(71) Applicant: BASF SE, Ludwigshafen am Rhein (DE)

(72) Inventors: Karl HAEBERLE, Speyer (DE); Thomas SCHMIDT, Ludwigshaven am Rhein (DE); Alexander PANCHENKO, Ludwigshafen am Rhein (DE)

(21) Appl. No.: 17/050,744

(22) PCT Filed: Apr. 15, 2019
(86) PCT No.: PCT/EP2019/059586

§ 371 Date: Oct. 26, 2020

(30) Foreign Application Priority Data
Apr. 25, 2018 (EP) 18169299.7

(51) Int. Cl. C08G 18/12; C08G 18/08; C08G 18/32; C08G 18/42; C08G 18/75; C08G 18/72; C08L 75/06

(52) U.S. Cl. CPC C08G 18/12; C08G 18/0823; C08G 18/3206; C08G 18/3228; C08G 18/3234; C08L 2201/50; C08G 18/755; C08G 18/758; C08G 18/722; C08L 75/06; C08G 18/4238

(57) ABSTRACT

An aqueous dispersion of a polyurethane wherein the polyurethane is obtained by reacting di- or polyfunctional cyanates, diols, a dicarboxylate with one or two secondary amino groups and optionally further compounds.

The present invention relates to an aqueous dispersion of a polyurethane wherein the polyurethane is obtained by reacting di- or polyfunctional isocyanates, diols, a dicarboxylate with one or two secondary amino group and optionally further compounds.

Aqueous polyurethane dispersions are often produced industrially using the process known as the “prepolymer mixing technique”. In that process polyurethanes are first prepared in an organic solvent and the resulting polyurethane solution is subsequently dispersed in water to obtain an aqueous polyurethane dispersion. Hydrophilic groups are required to make the polyurethane dispersible in water. One possibility is to add emulsifiers or protective colloids. However, such emulsifiers and protective colloids may have a negative impact on the properties of the polyurethane in technical applications. Emulsifiers and protective colloids tend to migrate as they are not bonded to the polyurethane.

Another possibility is to introduce hydrophilic groups into the polyurethane itself by copolymerizing compounds that have isocyanate groups or functional groups reactive with isocyanate and, in addition, a hydrophilic group. DE-A 2035732 discloses the copolymerization of compounds with amino groups and a sulfonic acid group to obtain a polyurethane that is dispersible in water. From DE-A 2034479 polyurethanes are known which are dispersible in water due to a content of compound with one carboxylic group. Such compounds are obtained by reacting unsaturated carboxylic acids, such as acrylic acid, with a primary diamine. The unsaturated group adds to the primary amino group which is known as Michael addition reaction. The obtained product of the Michael addition reaction comprises one carboxylic group, one secondary amino group and one unreacted primary amino group.

It was an object of this invention to provide polyurethane dispersions which high stability of the dispersed polyurethane particles. The process to prepare the polyurethane dispersion should be easy to perform and should have high efficiency. In particular, it is desired to use less hydrophilic comonomers in the polymerization. With less hydrophilic comonomers more comonomers may be used that contribute to the mechanical properties of the polyurethane.

Accordingly, the polyurethane dispersion described above and a process for the preparation of the polyurethane dispersion have been found.

To the dicarboxylate with at least one secondary amino group

The polyurethane is obtained by reacting di- or polyfunctional isocyanates, diols, a dicarboxylate with one or two secondary amino group and optionally further compounds.

The dicarboxylate comprises one or two secondary amino group. Preferably, the dicarboxylic acid comprises two secondary amino groups.

Preferably, the dicarboxylate is a compound of formula I

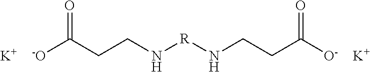

wherein R represents a bond or a hydrocarbon group with 1 to 20 carbon atoms and K⁺ represents an inorganic or organic cation.

Preferably, R represents a hydrocarbon group with 2 to 16, notably 2 to 10 carbon atoms.

Preferably, R is a non-aromatic hydrocarbon group.

More preferably, R represents an aliphatic hydrocarbon group comprising a cycloaliphatic ring system.

Preferably, K⁺ represents a metal cation or a quaternary ammonium cation. The quaternary ammonium cation is preferably formed by reacting a tertiary amino compound with the corresponding dicarboxylic acid of the dicarboxylate. The tertiary amino compound is preferably an amino compound substituted by three organic groups selected from alkyl or hydroxy alkyl groups, notably from C1- to C8-alkyl or C1- to C8-hydroxyalkyl groups.

More preferably, K⁺ is the cation of an alkali metal, notably of sodium or kalium.

In a most preferred embodiment of the invention, the dicarboxylate is a compound of formula II

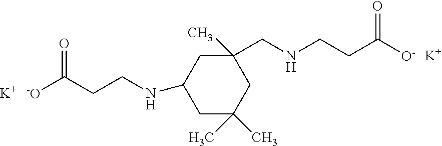

Dicarboxylates with one or two secondary amino group may be synthesized, for example by reacting

X) a compound with a double bond and a carboxylate group or a group that can be transferred into a carboxylate group

With

Y) a compound comprising two primary amino groups.

Preferably, X) is a compound with a double bond and a group that can be transferred into a carboxylate group. The group which can be transferred into a carboxylate group is preferably a nitrile group.

The obtained compound comprises X) and Y) in a molar ratio of X):Y) of 2:1. In the synthesis X) or Y) may be used in excess. Preferably, Y) is used in excess. The product is a Michael adduct.

In a preferred embodiment compound X) is acrylonitrile and the adduct formed by reacting X) and Y) is the compound of formula III

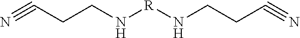

wherein R has the same meaning as in formula I

Compounds Y) are, for example, diaminoethane, diaminopropane, diaminobutane, diaminohexane, amino-3-aminomethyl-3,5,5-trimethyl-cyclohexane (known as isophorondiamine, shortly IPDA), 4,4′-diaminodicyclohexylmethane, 4,4′-diamino-3,3′-dimethyldicyclohexylmethane, 1,4-diaminocyclohexane, hydrazine which may be used also in form of its hydrate.

Preferred compounds Y) are amino-3-aminomethyl-3,5,5-trimethyl-cyclohexane (isophorondiamine, IPDA) and 4,4′-diaminodicyclohexylmethane, whereby IPDA is most preferred.

The adduct of 2 mols of acrylonitrile and one mol IPDA is known as Baxxodur® PC 136 which is available from BASF.

The compound of formula III is easily transferred into the compound of formula I by saponification of the two nitrile groups. By saponification the nitrile groups become carboxylate groups.

Preferably, the saponification is performed by reacting the compound of formula III with a base, notably with an alkali hydroxide.

To the polyurethane

The polyurethane is obtained by reacting di- or polyfunctional isocyanates, diols, a dicarboxylic acid with at least one secondary amino group and optionally further compounds.

Specifically, the polyurethane is obtained by reacting
a) di- or polyfunctional isocyanates with 4 to 30 carbon atoms
b) diols of which
b.1) 10 to 100 mol %, based on the total amount of diols (b), have a molecular weight of from 500 to 5000 and
b.2) 0 to 90 mol %, based on the total amount of diols (b), have a molecular weight of from 60 to 500 g/mol,
c) the dicarboxylate with one or two secondary amino groups and
d) optionally compounds with at least one isocyanate group or at least one isocyanato-reactive group which are different from compounds a), b) and c)

Di-functional isocyanates are compounds with two isocyanate groups, also referred to as diisocyanates.

Poly-functional isocyanates are compounds with at least three isocyanate groups, also referred to as polyisocyanates.

The di- or polyfunctional isocyanates may be aliphatic, aromatic and cycloaliphatic compounds which includes compounds that have combinations of (cyclo)aliphatic or aromatic groups.

Preferably, the di- or polyfunctional isocyanates do not comprise any other constituents than the isocyanate groups and hydrocarbon groups.

Usually a mixture of different di- or polyfunctional isocyanates is used. The NCO functionality of the mixture is preferably from 2 to 4.

The diisocyanates are preferably isocyanates having 4 to 20 carbon atoms. Examples of customary diisocyanates are aliphatic diisocyanates such as tetramethylene diisocyanate, hexamethylene diisocyanate (1,6-diisocyanatohexane), octamethylene diisocyanate, decamethylene diisocyanate, dodecamethylene diisocyanate, tetradecamethylene diisocyanate, esters of lysine diisocyanate, tetramethylxylylene diisocyanate, trimethylhexane diisocyanate or tetramethylhexane diisocyanate, cycloaliphatic diisocyanates such as 1,4-, 1,3- or 1,2-diisocyanatocyclohexane, trans/trans, the cis/cis and the cis/trans isomer of 4,4′- or 2,4′-di(isocyanatocyclohexyl)methane, 1-isocyanato-3,3,5-trimethyl-5-(isocyanatomethyl)cyclohexane (isophorone diisocyanate), 2,2-bis(4-isocyanatocyclohexyl)propane, 1,3- or 1,4-bis(isocyanatomethyl)cyclohexane or 2,4- or 2,6-diisocyanato-1-methylcyclohexane, and aromatic diisocyanates such as 2,4- or 2,6-toluylene diisocyanate and the isomer mixtures thereof, m- or p-xylylene diisocyanate, 2,4′- or 4,4′-diisocyanatodiphenylmethane and the isomer mixtures thereof, 1,3- or 1,4-phenylene diisocyanate, 1-chloro-2,4-phenylene diisocyanate, 1,5-naphthylene diisocyanate, diphenylene 4,4′-diisocyanate, 4,4′-diisocyanato-3,3′-dimethylbiphenyl, 3-methyldiphenylmethane 4,4′-diisocyanate, 1,4-diisocyanatobenzene or diphenyl ether 4,4′-diisocyanate.

Suitable diisocyanates are also the uretdiones of the above diisocyanates.

Mixtures of said diisocyanates may also be used.

Preference is given to aliphatic and cycloaliphatic diisocyanates, and particular preference to isophorone diisocyanate, hexamethylene diisocyanate, meta-tetramethylxylylene diisocyanate (m-TMXDI) and 1,1-methylenebis[4-isocyanato]cyclohexane (H₁₂MDI).

Suitable polyisocyanates include polyisocyanates containing isocyanurate groups, polyisocyanates containing biuret groups, polyisocyanates containing urethane groups or allophanate groups, polyisocyanates containing oxadiazinetrione groups, uretonimine-modified polyisocyanates of linear or branched C₄-C₂₀-alkylene diisocyanates, cycloaliphatic diisocyanates having 6 to 20 carbon atoms in all or aromatic diisocyanates having 8 to 20 carbon atoms in all, or mixtures thereof.

The diisocyanates and polyisocyanates which can be used preferably have an isocyanate group (calculated as NCO, molecular weight=42) content of from 10 to 60% by weight based on the diisocyanate and polyisocyanate (mixture), more preferably from 15 to 60% by weight and very preferably from 20 to 55% by weight.

Preference is given to aliphatic and/or cycloaliphatic diisocyanates and polyisocyanates, examples being the abovementioned aliphatic and cycloaliphatic diisocyanates and polyisocyanates or mixtures thereof.

Diols b.1) have a molecular weight of from 500 to 5000 g/mol, preferably from about 1000 to 3000 g/mol.

The diols b.1) are, in particular, polyesterdiols. Such polyesterdiols are preferably obtained by reacting dihydric alcohols with dibasic carboxylic acids. Instead of the free polycarboxylic acids it is also possible to use the corresponding polycarboxylic anhydrides or corresponding polycarboxylic esters of lower alcohols, or mixtures thereof, to prepare the polyesterpolyols. The polycarboxylic acids can be aliphatic, cycloaliphatic, araliphatic, aromatic or heterocyclic and can be unsubstituted or substituted, by halogen atoms, for example, and/or saturated or unsaturated. Examples are suberic, azelaic, phthalic, and isophthalic acid, tetrahydrophthalic, hexahydrophthalic, tetrachlorophthalic, endomethylenetetrahydrophthalic, glutaric and maleic anhydride, maleic acid, fumaric acid and dimeric fatty acids. Preference is given to dicarboxylic acids of the general formula HOOC—(CH₂)_{y}—COOH, where y is a number from 1 to 20, preferably an even number from 2 to 20, examples being succinic, adipic, sebacic and dodecanedicarboxylic acids.

Examples of suitable dihydric alcohols are ethylene glycol, 1,2-propanediol, 1,3-propanediol, 1,3-butanediol, 1,4-butenediol, 1,4-butynediol, 1,5-pentanediol, neopentyl glycol, bis(hydroxymethyl)cyclohexanes such as 1,4-bis(hydroxymethyl)cyclohexane, 2-methyl-1,3-propanediol and also diethylene glycol, triethylene glycol, tetraethylene glycol, polyethylene glycol, dipropylene glycol, polypropylene glycol, dibutylene glycol and polybutylene glycols. Preference is given to neopentylglycol and alcohols of the general formula HO—(CH₂)ₓ—OH, where x is a number from 1 to 20, preferably an even number from 2 to 20. Examples of such alcohols are ethylene glycol, 1,4-butanediol, 1,6-hexanediol, 1,8-octanediol and 1,12-dodecanediol.

Also suitable are polycarbonatediols, lactone-based polyesterdiols.

Further suitable diols b.1) are polyetherdiols. They are obtainable in particular by addition polymerization of ethylene oxide, propylene oxide, butylene oxide, tetrahydrofuran, styrene oxide or epichlorohydrin with itself, in the presence, for example, of BF₃, or by addition reaction of these compounds, alone or in a mixture or in succession, onto starter components containing reactive hydrogens, such as alcohols or amines, examples being water, ethylene glycol, 1,2-propanediol, 1,3-propanediol, 2,2-bis(4-hydroxydiphenyl)propane or aniline. Particular preference is given to polytetrahydrofuran having a molecular weight of from 500 to 5000 g/mol and, in particular, from 1000 to 4500 g/mol.

Mixtures of different diols b.1) may be used.

It is possible to employ as diols b) not only the diols b.1) but also low molecular mass diols b.2) having a molecular weight of from about 50 to 500, preferably from 60 to 200 g/mol.

Compounds employed as monomers (b2) are notably short-chain alkanediols cited above for the preparation of polyesterpolyols, preference being given to the unbranched diols having from 2 to 12 carbons and an even number of carbons, and to 1,5-pentanediol and neopentyl glycol.

The proportion of the diols (b1), based on the total amount of diols (b), is preferably from 10 to 100 mol %, and the proportion of the diols (b2), based on the total amount of diols (b), is preferably from 0 to 90 mol %.

Compounds c) is the dicarboxylate with one or two secondary amino group, preferably the compound of formula I and most preferably the compound of formula II.

The term “the dicarboxylate with one or two secondary amino groups” shall include mixtures of dicarboxylates with one or two secondary amino groups. In a preferred embodiment compound c) is only one specific dicarboxylate with one or two secondary amino groups.

Preferably, the polyurethane comprises the dicarboxylate with one or two secondary amino groups in an amount of 0.01 to 0.5 mol per 1 kg polyurethane.

More preferably, the polyurethane comprises the dicarboxylate with one or two secondary amino groups in an amount of 0.05 to 0.3 mol per 1 kg polyurethane.

Most preferably, the polyurethane comprises the dicarboxylate with one or two secondary amino groups in an amount of 0.08 to 0.25 mol per 1 kg polyurethane.

Compounds d) are optional. Compounds d) include any compound that has at least one isocyanate group or at least one isocyanate reactive group, such as a hydroxy, thiol or primary or secondary amino group but does not fall under the definition of compounds a), b) and c).

Such compounds are, for example, compounds d.1) having at least at two primary or at least two secondary amino group or having at least one primary and at least one secondary amino group but having no additional hydrophilic group. Compounds d.1) may be used as chain extenders or crosslinkers.

Such compounds are, for example, compounds d.2) having with at least three hydroxy groups but no additional hydrophilic group. Such compounds may be used as crosslinkers.

Such compounds are, for example, compounds d.3) with at least one isocyanate group or at least one isocyanato-reactive group and additionally at least one hydrophilic group or one potentially hydrophilic group which can be transferred into a hydrophilic group. Like compound c), compounds d.3) have the ability to render the polyurethanes dispersible in water.

The hydrophilic groups of d.3) can be nonionic or, preferably, ionic, i.e., cationic or anionic groups. A potentially hydrophilic group is a group which can be easily transformed into a hydrophilic group when it is intended to disperse the polyurethane in water. Such potentially hydrophilic groups are notably acid groups than can be transformed into hydrophilic groups by salt formation or tertiary amino groups that can be transformed into ionic groups by reacting them with an acid or by quaternization with alkylating agents.

In a preferred embodiment, compounds d.3) are used in small amounts, only, if at all. Preferably compounds d.3) are used in an amount of less than 50% by weight, more preferably of less than 20% by weight, notably of less than 5% by weight based on the total amount of compounds c) and d.3).

In a most preferred embodiment, compounds d.3) are not used at all.

Further compounds d) are, for example, compounds d.4) with one isocyanate group or one isocyanato-reactive group without hydrophilic groups or potentially hydrophilic groups. As such compounds have only one reactive group, they may be used to limit the molecular weight of the polyurethane by chain termination. Examples are methanol, ethanol, isopropanol, n-propanol, n-butanol, isobutanol, sec-butanol, tert-butanol, ethylene glycol monomethyl ether, ethylene glycol monoethyl ether, 1,3-propanediol monomethyl ether, n-hexanol, n-heptanol, n-octanol, n-decanol, n-dodecanol (lauryl alcohol) and 2-ethylhexanol.

If used at all, compounds d.4) are usually used in minor amounts, such as amounts of less than 10 mol, notably less than 1 mol per 100 mols of compounds b).

Compounds a), b), c) and d) are preferably reacted in molar amounts such that the ratio A:B, whereby
A) is the molar amount of isocyanate groups and
B) is the sum of the molar amount of the hydroxyl groups and of all other functional groups which are able to react with isocyanates in an addition reaction,
   is from 0.5:1 to 2:1, preferably from 0.8:1 to 1.5 and more preferably from 0.9:1 to 1.2:1. With very particular preference the ratio A:B is as close as possible to 1:1.

The reaction of compounds a) to d) is a well know polyaddition reaction. The polyaddition reaction is preferably performed at temperatures of 20 to 180° C., preferably 50 to 150° C. under atmospheric pressure.

The reaction times required normally extend from a few minutes to several hours. It is known within the field of polyurethane chemistry how the reaction time is influenced by a multiplicity of parameters such as temperature, monomer concentration and monomer reactivity.

For accelerating the reaction, it is possible to use catalysts. Suitable catalysts are commonly known.

These are, for example, organic amines, particularly tertiary aliphatic, cycloaliphatic or aromatic amines, and/or Lewis-acidic organometallic compounds.

Preferred Lewis-acidic organometallic compounds are dimethyltin diacetate, dibutyltin dibutyrate, dibutyltin bis(2-ethylhexanoate), dibutyltin dilaurate, dioctyltin dilaurate, zirconium acetylacetonate and zirconium 2,2,6,6-tetramethyl-3,5-heptanedionate.

Bismuth, cobalt or cesium based catalysts may be used as well.

Suitable apparatus for the polyaddition reaction includes stirred tanks, particularly when solvents are used to ensure a low viscosity and effective heat removal.

If the reaction is carried out in bulk suitable equipment, because of the generally high viscosities and the generally short reaction times, includes in particular extruders, especially self-cleaning multiscrew extruders.

The polyurethane and the aqueous dispersion thereof are preferably obtained by a process wherein
I) compounds a), b), c) and optionally d) are reacted to a polyurethane in an organic solvent and
II) the polyurethane is dispersed in water, whereby
   components d) having primary or secondary amino groups as isocyanato-reactive groups may be reacted in I) as well or may be added partially or totally during or after step II.

The organic solvent used in step I may be any solvent which solves compounds a) to e). Preferred solvents have high miscibility with water, notably unlimited miscibility with water. Preferably, the organic solvent used has a boiling point of at maximum 100° C. (normal pressure). A preferred solvent is, for example, acetone.

The organic solvent may be totally removed from the aqueous dispersion, if desired. Alternatively, the solvent may remain totally or partially in the aqueous dispersion.

In a preferred embodiment no additional emulsifiers or protective colloids are used to disperse the polyurethane in water. Hence, the preferred aqueous dispersion of the polyurethane obtained according to this invention is free of emulsifiers and protective colloids.

The average particle size (z-average) as measured by means of dynamic light scattering with the Malvern® Autosizer 2 C of the obtained polyurethane particles dispersed in water is generally <1000 nm, preferably <500 nm, more preferably <200 nm and very preferably between 20 and below 200 nm.

The dispersions generally have a solids content of from 10 to 75%, preferably from 20 to 65%, by weight and a viscosity of from 10 to 500 mPas (measured at a temperature of 20° C. and at a shear rate of 250 s⁻¹).

The dispersions prepared in accordance with the invention may additionally be mixed with other components typical for the cited applications, examples being surfactants, detergents, dyes, pigments, colour transfer inhibitors and optical brighteners.

The dispersions can be subjected to physical deodorization, if desired, following their preparation.

Physical deodorization may involve stripping the dispersion using steam, an oxygen-containing gas, preferably air or nitrogen, for example, a stirred vessel, as described in DE-B 12 48 943, or in a counter current column, as described in DE-A 196 21 027.

The aqueous polyurethane formulations of the invention are suitable for coating and bonding substrates. They are notably suitable for coating or adhesively bonding wood, wood veneer, paper, paperboard, cardboard, textile, leather, nonwoven, plastics surfaces, glass, ceramic, mineral building materials, uncoated metals or coated metals.

They find application, for example, in the production of films or thin sheets, for impregnating textiles or leather, as dispersants, as pigment grinding agents, as primers, as adhesion promoters, as hydrophobicizers, as a laundry detergent additive or as an additive to cosmetic formulations, or for producing mouldings or preparing hydrogels.

In the context of their use as coating materials the polyurethane dispersions can be employed in particular as primers, surfacers, pigmented topcoat materials and clearcoat materials in the automotive refinishing or large-vehicle finishing sector. The coating materials are especially suitable for applications that call for particularly high application reliability, exterior weathering stability, optical qualities, solvent resistance, chemical resistance and water resistance, such as in automotive refinish and large-vehicle finishing.

The aqueous dispersion of the polyurethanes obtained according to this invention is homogeneous and has a high stability. No additional emulsifiers or protective colloids are required. Due to the presence of two carboxylic acid groups in compound c) less compounds with hydrophilic groups are required for the preparation of the polyurethane. The polyurethane has good mechanical properties.

### EXAMPLES

| | |
|---|---|
| IPDI | isophorone diisocyanate |
| H12MDI | 1,1-methylenebis[4-isocyanato]cyclohexane |
| Compound II | compound of formula II |
| DETA | diethylene triamine |
| PUD | polyurethane dispersion |
| Baxxodur ®PC 136 | adduct of two mols of acrylonitrile and IPDA |

### Example 1

### Preparation of Compound II

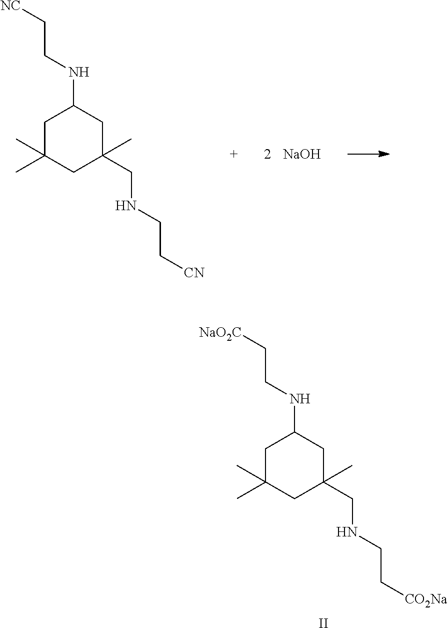

A 2-litre stirred flask was charged with 469.0 g of sodium hydroxide (20% by weight aqueous solution, 2.35 mol). Afterwards 197.8 g of 40% by weight aqueous solution of compound II was added to accelerate the desired saponification reaction of the starting material which is the corresponding dinitrile. The saponification reaction is also possible without the addition of compound II, but the dosage time has to be prolonged, especially at the beginning of the dosage.

The temperature of the resulting mixture was raised to 90° C. Then, 310.2 g Baxxodur® PC136 (1.12 mol) was dosed within 180 min. by keeping the reaction temperature at 90−95° C. The dosage was operated with a jacket dropping funnel which was heated to 55° C. to reduce the viscosity of compound I. After completion of the nitrile addition the dropping funnel was flushed with 102.0 g of de-ionized water and the reaction mixture was stirred at 95−100° C. for additional 120 min. to finalize the reaction and to remove the ammonia formed during saponification. A clear solution was obtained. After cooling to 20° C. addition de-ionized water was added to adjust the dry matter content to 43%.

The final product was characterized by NMR (1H- and 13C-NMR).

### Example 2

Preparation of a PUD with Compound II

600 g (0.30 mol) of a polyester diol made from adipic acid, neopentylglycol and 1,6-hexanediol (molar ratio 1/1) (OH-number 56), 26,7 g (0.014 mol) of a butanol-started polyethylene oxide, 100 g acetone and 0,3 g dibutyl tin dilaurate were charged to a vessel and stirred at 83° C. Then 89.8 g (0.404 mol) IPDI and 106.7 g (0.404 mol) H12MDI were added followed by stirring of the obtained mixture at 83° C. for 60 minutes. Then 27.0 g (0.30 mol) 1,4-butane diol were added and the reaction continued for further 120 minutes. The mixture was diluted by adding 850 g acetone. The NCO-content of the obtained mixture was determined to be 0.91% by weight (calculated: 0.93% by weight). Thereafter 73.1 g (0.10 mol of Compound II) of the solution of Example 1 were added. After 10 minutes 1300 g of water were added within 15 minutes. Then a mixture of 5.8 g (0.0562 mol) DETA and 2.7 g (0.0159 mol) IPDA were added. Finally, acetone was removed by distillation at reduced pressure.

A homogeneous polyurethane dispersion with small polyurethane particles was obtained. The polyurethane dispersion had a solids content of 36,0% by weight.

### Comparison Example

Preparation of a PUD with diamino-monocarboxylic acid (analogous to the disclosure of DE-A 2 034 479)

600 g (0.30 mol) of a polyester diol made from adipic acid, neopentylglycol and 1,6-hexanediol (molar ratio 1/1) (OH-number 56), 26,7 g (0.014 mol) of a butanol-started polyethylene oxide, 100 g acetone and 0,3 g dibutyl tin dilaurate were charged to a vessel and stirred at 83° C. Then 89.8 g (0.404 mol) IPDI and 106.7 g (0.404 mol) H12MDI were added followed by stirring of the obtained mixture at 83° C. for 60 minutes. Then 27.0 g (0.30 mol) 1,4-butane diol were added and the reaction continued for further 120 minutes. The mixture was diluted by adding 850 g acetone. The NCO-content of the obtained mixture was determined to be 0.93% by weight (calculated: 0.93% by weight). Thereafter 42.0 g (0.10 mol) of the adduct of sodium acrylate to ethylene diamine were added. After 10 minutes 1300 g of water were added within 15 minutes. Then a mixture of 5.8 g (0.0562 mol) DETA and 2.7 g (0.0159 mol) IPDA were added. Finally, acetone was removed by distillation at reduced pressure.

A homogeneous polyurethane dispersion with small polyurethane particles was obtained. The polyurethane dispersion had a solids content of 38,0% by weight.

1.-11. (canceled)

12. A synthesis of a dicarboxylate with one or two secondary amino groups wherein the dicarboxylate is a compound of Formula I 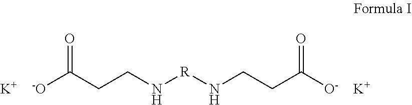
wherein R represents a bond or a hydrocarbon group with 1 to 20 carbon atoms and K⁺ represents an inorganic or organic cation,
comprising reacting
X) acrylonitrile with
Y) a compound comprising two primary amino groups,
wherein the adduct formed by the by reacting X) and Y) is a compound of Formula III 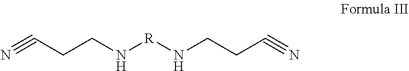
wherein R has the same meaning as in Formula I,
wherein the compound of Formula III is transferred into the compound of Formula I by saponification of the two nitrile groups.

13. The synthesis according to claim 12 wherein the saponification is performed by reacting the compound of Formula III with a base.

14. The synthesis according to claim 13 wherein the base is an alkali hydroxide.

15. The synthesis according claim 12 wherein R represents a hydrocarbon group with 2 to 16 carbon atoms.

16. The synthesis according claim 12 wherein R represents a hydrocarbon group with 2 to 10 carbon atoms.

17. The synthesis according claim 12 wherein R is a non-aromatic hydrocarbon group.

18. The synthesis according claim 12 wherein the compound Y) is diaminoethane, diaminopropane, diaminobutane, diaminohexane, amino-3-aminomethyl-3,5,5-trimethylcyclohexane, 4,4′-diaminodicyclohexylmethane, 4,4′-diamino-3,3′-dimethyldicyclohexylmethane, 1,4-diaminocyclohexane, or hydrazine.

19. The synthesis according claim 12 wherein K⁺ represents a metal cation or a quaternary ammonium cation.

20. The synthesis according claim 12 wherein K⁺ is the cation of an alkali metal.

21. The synthesis according claim 12 wherein the compound Y) is used in excess.

22. The synthesis according claim 12 wherein by the saponification the nitrile groups become carboxylate groups.
